(19) United States
(12) Patent Application Publication
(10) Pub. No.: US 2025/0241611 A1
(43) Pub. Date: Jul. 31, 2025

(54) INFORMATION PROCESSING APPARATUS, INFORMATION PROCESSING METHOD, INFORMATION PROCESSING PROGRAM, AND MEDICAL IMAGE CAPTURING SYSTEM

(71) Applicant: FUJIFILM Corporation, Tokyo (JP)

(72) Inventor: Ryosuke TSUCHIDA, Tokyo (JP)

(73) Assignee: FUJIFILM Corporation, Tokyo (JP)

(21) Appl. No.: 19/037,483

(22) Filed: Jan. 27, 2025

(30) Foreign Application Priority Data
Jan. 31, 2024 (JP) 2024-012902

(51) Int. Cl. A61B 6/00; A61B 6/04

(52) U.S. Cl. CPC A61B 6/547; A61B 6/04

(57) ABSTRACT

An information processing apparatus acquires at least one of examination information regarding an examination, subject information regarding a subject, or operator information regarding an operator and derives a movement speed of the patient table between an initial position and an imaging position based on the acquired information.

### CROSS-REFERENCE TO RELATED APPLICATIONS

This application claims priority from Japanese Patent Application No. 2024-012902, filed on Jan. 31, 2024, the entire disclosure of which is incorporated herein by reference.

### BACKGROUND

### 1. Technical Field

The present disclosure relates to an information processing apparatus, an information processing method, an information processing program, and a medical image capturing system.

### 2. Description of the Related Art

In recent years, with advancements in medical devices such as a computed tomography (CT) apparatus and a magnetic resonance imaging (MRI) apparatus, higher quality and high-resolution three-dimensional images have been used for image diagnosis. In a medical image capturing apparatus such as a CT apparatus and an MRI apparatus, a subject is placed on a patient table at an initial position, the patient table with the subject placed thereon is moved to an imaging position (for example, a position immediately in front of a gantry), and then capturing of a medical image is started.

JP2014-054388A discloses a technology for allowing an operator to input a movement direction and a movement speed of a patient table via an input device such as a touch panel.

JP2020-068879A discloses a technology for assisting an operator in moving a patient table by controlling a movement speed of the patient table according to a force applied by the operator to a top surface of the patient table.

### SUMMARY

In the above-mentioned medical image capturing apparatus, automatically moving the patient table between the initial position and the imaging position can improve the efficiency of image capturing. In this case, increasing the movement speed of the patient table can shorten the image capturing time, which is preferable. However, the optimal movement speed of the patient table varies depending on various factors such as the examination, the subject, and the operator.

The present disclosure has been made in view of the above circumstances, and an object of the present disclosure is to provide an information processing apparatus, an information processing method, an information processing program, and a medical image capturing system capable of controlling a movement speed of a patient table on which a subject is placed in a medical image capturing apparatus to an appropriate speed.

According to a first aspect, there is provided an information processing apparatus that derives a movement speed of a patient table of a medical image capturing system which captures a medical image of a subject placed on the patient table, the information processing apparatus comprising: at least one processor, in which the processor is configured to: acquire at least one of examination information regarding an examination, subject information regarding the subject, or operator information regarding an operator; and derive a movement speed of the patient table between an initial position and an imaging position based on the acquired information.

According to a second aspect, in the information processing apparatus according to the first aspect, the processor may be configured to control movement of the patient table between the initial position and the imaging position in accordance with the derived movement speed.

According to a third aspect, in the information processing apparatus according to the first or second aspect, the examination information may include a type of examination.

According to a fourth aspect, in the information processing apparatus according to the third aspect, the processor may be configured to, in a case in which the type of examination indicates an examination performed by connecting a medical device to the subject, derive the movement speed of the patient table to be lower than in a case in which the type of examination indicates an examination performed without connecting the medical device to the subject.

According to a fifth aspect, in the information processing apparatus according to the first or second aspect, the subject information may include at least one of information associated with identification information of the subject, biological information of the subject, a voice uttered by the subject placed on the patient table, or an optical image obtained by imaging the subject placed on the patient table.

According to a sixth aspect, in the information processing apparatus according to the fifth aspect, the processor may be configured to derive a movement speed of the patient table before start of movement of the patient table and then derive a movement speed that has been adjusted for the derived movement speed of the patient table based on at least one of the biological information, the voice, or the optical image acquired during the movement of the patient table.

According to a seventh aspect, in the information processing apparatus according to the first or second aspect, the operator information may include at least one of information associated with identification information of the operator, a voice uttered by the operator, or an optical image obtained by imaging the operator.

According to an eighth aspect, in the information processing apparatus according to the seventh aspect, the processor may be configured to derive a movement speed of the patient table before start of movement of the patient table and then derive a movement speed that has been adjusted for the derived movement speed of the patient table based on at least one of the voice or the optical image acquired during the movement of the patient table.

According to a ninth aspect, there is provided an information processing method comprising: causing a processor of an information processing apparatus that includes at least one processor and that derives a movement speed of a patient table of a medical image capturing system which captures a medical image of a subject placed on the patient table, to execute: acquiring at least one of examination information regarding an examination, subject information regarding the subject, or operator information regarding an operator; and deriving a movement speed of the patient table between an initial position and an imaging position based on the acquired information.

According to a tenth aspect, there is provided an information processing program for causing a processor of an information processing apparatus that includes at least one processor and that derives a movement speed of a patient table of a medical image capturing system which captures a medical image of a subject placed on the patient table, to execute: acquiring at least one of examination information regarding an examination, subject information regarding the subject, or operator information regarding an operator; and deriving a movement speed of the patient table between an initial position and an imaging position based on the acquired information.

According to an eleventh aspect, there is provided a medical image capturing system comprising: a patient table on which a subject is placed; a medical image capturing apparatus that captures a medical image of the subject; and the information processing apparatus according to any one of the first to eighth aspects, the information processing apparatus deriving a movement speed of the patient table.

According to the present disclosure, it is possible to control the movement speed of the patient table on which the subject is placed in the medical image capturing apparatus to an appropriate speed.

### BRIEF DESCRIPTION OF THE DRAWINGS

FIG. 1 is a perspective view showing an outline of a CT apparatus.

FIG. 2 is a view of the CT apparatus as seen from a side.

FIG. 3 is a block diagram showing an example of a hardware configuration of an information processing apparatus.

FIG. 4 is a diagram showing an example of operator data.

FIG. 5 is a diagram showing an example of patient data.

FIG. 6 is a block diagram showing an example of a functional configuration of the information processing apparatus.

FIG. 7 is a flowchart showing an example of patient table movement processing.

FIG. 8 is a flowchart showing an example of initial movement speed derivation processing.

### DETAILED DESCRIPTION

Hereinafter, an example of an embodiment for implementing the technology of the present disclosure will be described in detail with reference to the drawings.

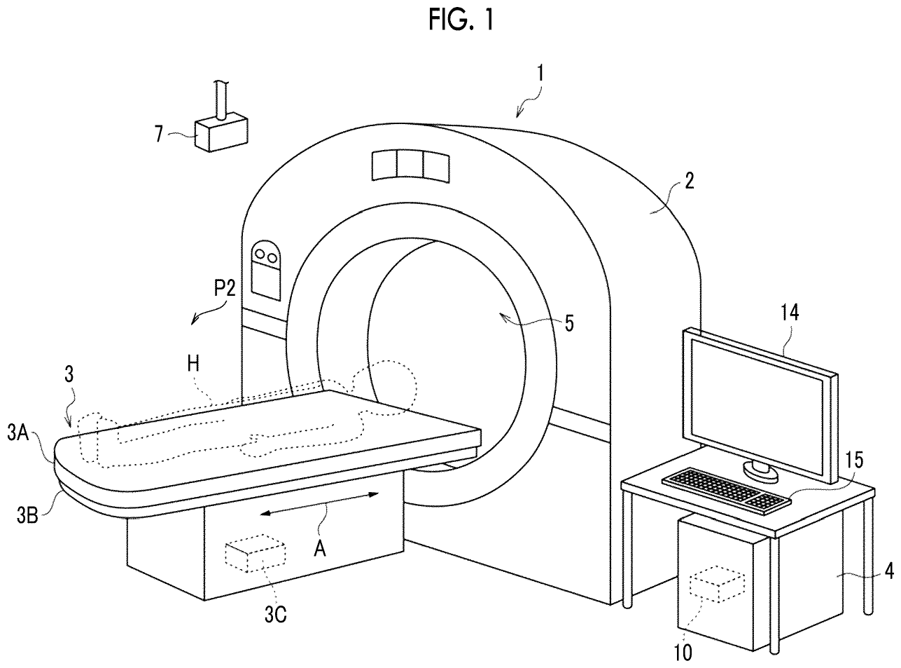

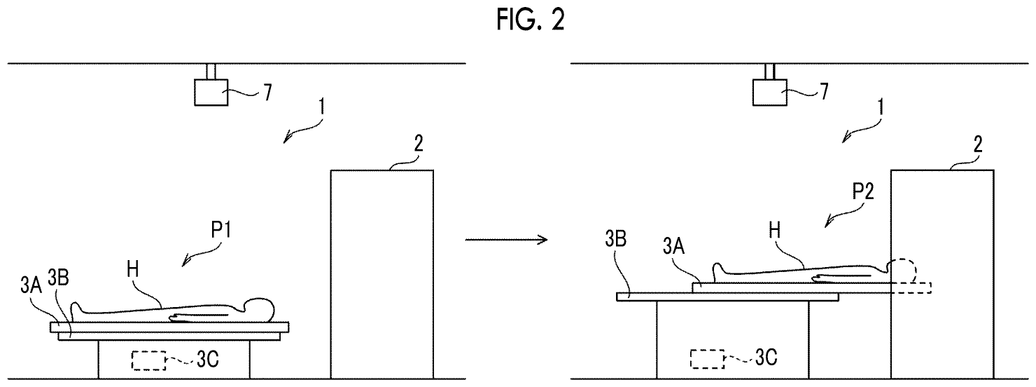

First, a configuration of a CT apparatus 1 will be described with reference to FIGS. 1 and 2. As shown in FIGS. 1 and 2, the CT apparatus 1 according to the present embodiment comprises a gantry 2, a patient table 3, and a console 4. The combination of the gantry 2, the patient table 3, and the console 4 is an example of a medical image capturing system according to the disclosed technology.

The gantry 2 has a tunnel-like structure including an opening portion 5 at the center thereof. A radiation source unit that emits X-rays and a detection unit that detects the X-rays to generate a radiation image are provided inside the gantry 2 (both not shown). The radiation source unit and the detection unit can rotate along an annular shape of the gantry 2 while maintaining a mutually opposing positional relationship. Additionally, a control unit that controls an operation of the CT apparatus 1 is provided inside the gantry 2. The gantry 2 is an example of a medical image capturing apparatus that captures a medical image of a subject.

A patient H as an example of the subject is placed on the patient table 3. The patient table 3 includes a patient table portion 3A on which the patient H lies, a base portion 3B that supports the patient table portion 3A, and a driving unit 3C that reciprocates the patient table portion 3A in a direction of an arrow A. The patient table portion 3A can slide with respect to the base portion 3B in the direction of the arrow A by the driving unit 3C. In a case in which a CT image as an example of the medical image is captured, the patient table portion 3A slides, thereby transporting the patient H lying on the patient table portion 3A into the opening portion 5 of the gantry 2.

As shown in FIG. 2, in a case in which the patient table 3 is at an initial position P1, the patient H lies on the patient table portion 3A. Thereafter, the patient table portion 3A is raised and slid by the driving unit 3C, thereby moving the patient table 3 to an imaging position P2. After the patient table portion 3A is slid to the imaging position P2, the imaging is started. In addition, after the end of the imaging, the patient table 3 is moved from the imaging position P2 to the initial position P1 by the driving unit 3C. The initial position P1 is a position different from the imaging position P2 and is, for example, a position farther from the gantry 2 and closer to a floor surface than the imaging position P2. Further, the imaging position P2 is a position at which the patient H is transported to the gantry 2 by sliding the patient table portion 3A in a case of capturing the CT image.

A camera 7 is installed on a ceiling above the patient table 3. The camera 7 is a camera that can capture a red (R), green (G), and blue (B) color image by detecting reflected light from the patient H. The camera 7 includes an imaging element such as a lens and a charge coupled device (CCD), and acquires a camera image which is a moving image by imaging the patient H on the patient table 3 at a predetermined frame rate and outputs the camera image to the console 4. The camera 7 may be a camera in which an RGB camera and a near infrared (NIR) camera are integrated. In this case, the NIR camera is a stereo camera, and information on the patient H in a depth direction can be acquired through parallax. By configuring the NIR camera of the camera 7 as the stereo camera, the information on the patient H on the patient table 3 in the depth direction can be acquired. The NIR camera can perform imaging even in a case in which an amount of light is insufficient. Therefore, in a case in which the brightness in an examination room is insufficient for the imaging with the RGB camera, the patient H may be imaged by the NIR camera. Additionally, the camera 7 may be provided on a wall surface, a housing of the gantry 2, or the like instead of on the ceiling. Further, the camera 7 may be provided at each of a plurality of locations.

The driving of the gantry 2, the driving of the patient table 3, and the imaging of the subject by the camera 7 are performed by an input from an operator such as a technician via the console 4. The console 4 includes an information processing apparatus according to the disclosed technology.

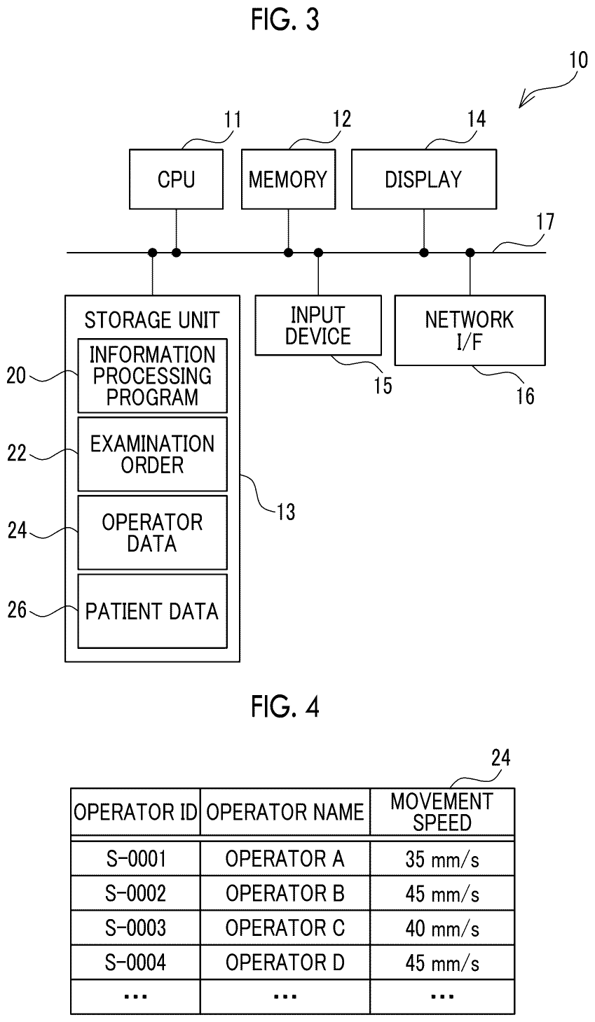

A hardware configuration of an information processing apparatus 10 according to the present embodiment will be described with reference to FIG. 3. Examples of the information processing apparatus 10 include computers such as a personal computer or a server computer. As shown in FIG. 3, the information processing apparatus 10 includes a central processing unit (CPU) 11, a memory 12 serving as a temporary storage area, and a non-volatile storage unit 13. In addition, the information processing apparatus 10 includes a display 14 such as a liquid crystal display, an input device 15 such as a keyboard and a mouse, and a network interface (I/F) 16 connected to the CT apparatus 1. The CPU 11, the memory 12, the storage unit 13, the display 14, the input device 15, and the network I/F 16 are connected to a bus 17. The CPU 11 is an example of a processor according to the disclosed technology. The display 14 and the input device 15 are also shown in FIG. 1.

The storage unit 13 is implemented by a hard disk drive (HDD), a solid state drive (SSD), a flash memory, or the like. The storage unit 13 serving as a storage medium stores an information processing program 20. The CPU 11 reads the information processing program 20 from the storage unit 13 and then expands the read information processing program 20 into the memory 12, and executes the expanded information processing program 20.

Additionally, the storage unit 13 stores an examination order 22, operator data 24, and patient data 26. The examination order 22 is an example of examination information regarding the examination. The examination order 22 includes a patient identifier (ID), which is identification information of the patient H to be examined using the CT apparatus 1, an operator ID, which is identification information of an operator who operates the CT apparatus 1, such as a radiologist, a type of examination, and the like. Examples of the type of examination include plain imaging, contrast imaging, and electrocardiogram-gated imaging. The plain imaging is an examination performed without connecting a medical device to the patient H. The contrast imaging and the electrocardiogram-gated imaging are examinations performed by connecting medical devices such as a catheter and an electrocardiogram sensor to the patient H.

The operator data 24 is an example of operator information regarding the operator. FIG. 4 shows an example of the operator data 24. As shown in FIG. 4, in the operator data 24, the operator ID, the operator name, and the movement speed of the patient table 3 are associated with each operator. The movement speed of the patient table 3 in the operator data 24 is a movement speed of the patient table 3 desired by the operator.

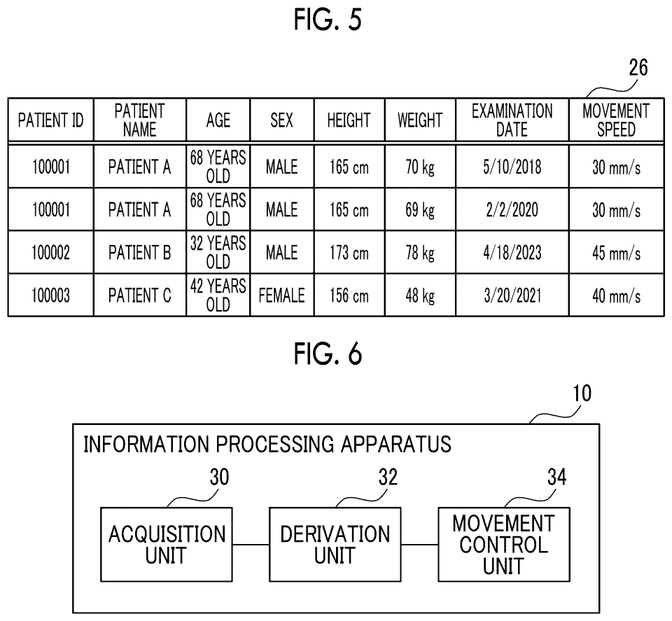

The patient data 26 is an example of subject information regarding the subject. FIG. 5 shows an example of the patient data 26. As shown in FIG. 5, in the patient data 26, the patient ID, the patient name, the patient age, the patient sex, the patient height, the patient weight, the examination date, and the movement speed of the patient table 3 are associated with each pair of the patient H and the examination date. The movement speed of the patient table 3 in the patient data 26 is a movement speed of the patient table 3 between the initial position P1 and the imaging position P2 in a case in which the examination using the CT apparatus 1 is performed on the patient H on the corresponding examination date.

The information processing apparatus 10 has a function of deriving the movement speed of the patient table 3 of the CT apparatus 1 that captures the medical image of the patient H placed on the patient table 3.

Next, a functional configuration of the information processing apparatus 10 will be described with reference to FIG. 6. As shown in FIG. 6, the information processing apparatus 10 includes an acquisition unit 30, a derivation unit 32, and a movement control unit 34. The CPU 11 executes the information processing program 20 to function as the acquisition unit 30, the derivation unit 32, and the movement control unit 34.

The acquisition unit 30 acquires the examination order 22, the operator data 24, and the patient data 26 from the storage unit 13. The examination order 22, the operator data 24, and the patient data 26 may be acquired from the memory 12 by being expanded into the memory 12 through the network I/F 16. In addition, the acquisition unit 30 sequentially acquires, from the camera 7, optical images (hereinafter, referred to as “patient images”) obtained by imaging the patient H placed on the patient table 3 with the camera 7 in accordance with a set frame rate. Further, the acquisition unit 30 acquires biological information of the patient H from a biological information sensor worn by the patient H. Examples of the biological information include electrocardiogram, pulse, respiration, and blood oxygen concentration. Furthermore, the acquisition unit 30 acquires a voice (hereinafter, referred to as a “patient voice”) uttered by the patient H via a voice input device (not shown) provided in an imaging room. The patient image, the biological information of the patient H, and the patient voice are examples of subject information regarding the subject.

Additionally, the acquisition unit 30 acquires an optical image (hereinafter, referred to as an “operator image”) obtained by imaging the operator with the camera 7. Further, the acquisition unit 30 acquires a voice (hereinafter, referred to as an “operator voice”) uttered by the operator via a voice input device provided in the imaging room. The operator image and the operator voice are examples of operator information regarding the operator.

The derivation unit 32 derives the movement speed of the patient table 3 between the initial position P1 and the imaging position P2 based on the examination order 22, the operator data 24, the patient data 26, the patient image, the biological information of the patient H, the patient voice, the operator image, and the operator voice acquired by the acquisition unit 30. The derivation unit 32 need not use all of these pieces of information. That is, the derivation unit 32 may derive the movement speed of the patient table 3 between the initial position P1 and the imaging position P2 based on one or a combination of two or more of the following: the examination order 22, the operator data 24, the patient data 26, the patient image, the biological information of the patient H, the patient voice, the operator image, and the operator voice. A specific example of derivation processing of the movement speed of the patient table 3 by the derivation unit 32 will be described below.

First, the derivation unit 32 derives the movement speed of the patient table 3 between the initial position P1 and the imaging position P2 based on the examination order 22, the operator data 24, and the patient data 26 before the start of the movement of the patient table 3 (that is, in a state in which the patient table 3 is located at the initial position P1). Hereinafter, the movement speed of the patient table 3 derived before the start of the movement of the patient table 3 will be referred to as an “initial movement speed”.

Specifically, in a case in which the patient ID included in the examination order 22 is present in the patient data 26, the derivation unit 32 sets a movement speed associated with the patient ID included in the patient data 26 as the initial movement speed. In a case in which a plurality of patient IDs included in the examination order 22 are present in the patient data 26, the derivation unit 32 may set a movement speed associated with the most recent examination date as the initial movement speed among a plurality of movement speeds associated with each patient ID. In addition, in a case in which the patient ID included in the examination order 22 is present in the patient data 26, the derivation unit 32 may set a movement speed higher than the movement speed associated with the patient ID as the initial movement speed. This is because it is estimated that the patient H having the patient ID is accustomed to the examination using the CT apparatus 1.

Additionally, in a case in which the patient ID included in the examination order 22 is not present in the patient data 26, that is, in a case in which the patient H having the patient ID receives the examination using the CT apparatus 1 for the first time, the derivation unit 32 refers to the operator data 24. In addition to this case, in a case in which the operator ID included in the examination order 22 is present in the operator data 24, the derivation unit 32 sets a movement speed associated with the operator ID included in the operator data 24 as the initial movement speed.

In a case in which the patient ID is not present in the patient data 26 and the operator ID is not present in the operator data 24, the derivation unit 32 sets a predetermined reference speed as the initial movement speed. The derivation unit 32 may vary the initial movement speed corresponding to the reference speed according to the age of the patient H. For example, in a case in which the age of the patient H is equal to or less than a first threshold value or equal to or greater than a second threshold value, the derivation unit 32 may set the initial movement speed to be lower than in a case in which the age of the patient H is greater than the first threshold value and less than the second threshold value. Examples of the first threshold value include a value defined as an upper limit value of an age range for young children, such as those of a lower age. Examples of the second threshold value include a value defined as a lower limit value of an age range for elderly individuals.

Next, in a case in which an examination type included in the examination order 22 indicates an examination performed by connecting a medical device to the patient H, the derivation unit 32 lowers the initial movement speed derived as described above. In this case, for example, the derivation unit 32 lowers the initial movement speed by a predetermined ratio such as 5% or by a predetermined speed such as 1 [mm/s]. That is, in a case in which the examination type indicates an examination performed by connecting the medical device to the patient H, the derivation unit 32 derives the movement speed of the patient table 3 to be lower than in a case in which the examination type indicates an examination performed without connecting the medical device to the patient H.

Further, the derivation unit 32 derives the initial movement speed of the patient table 3 before the start of the movement of the patient table 3 and then derives a movement speed that has been adjusted for the movement speed of the patient table 3 based on the patient image, the biological information of the patient H, the patient voice, the operator image, and the operator voice acquired by the acquisition unit 30 during the movement of the patient table 3.

For example, in a case in which the behavior of the patient H obtained by performing image analysis on the patient image corresponds to a behavior indicating that the patient H is anxious, such as the patient H thrashing about, the derivation unit 32 derives a movement speed that has been adjusted to be lower for the movement speed of the patient table 3. In addition, in a case in which the biological information of the patient H is outside a normal value range, such as the pulse being faster than normal, the derivation unit 32 derives a movement speed that has been adjusted to be lower for the movement speed of the patient table 3. Alternatively, in a case in which the patient voice corresponds to a voice indicating that the patient is anxious, the derivation unit 32 derives a movement speed that has been adjusted to be lower for the movement speed of the patient table 3.

Additionally, in a case in which the gesture of the operator obtained by performing image analysis on the operator image corresponds to a gesture that indicates increasing the movement speed, the derivation unit 32 derives a movement speed that has been adjusted to be higher for the movement speed of the patient table 3. Alternatively, in a case in which the gesture of the operator obtained by performing image analysis on the operator image corresponds to a gesture that indicates decreasing the movement speed, the derivation unit 32 derives a movement speed that has been adjusted to be lower for the movement speed of the patient table 3.

Further, in a case in which the operator voice corresponds to a voice that indicates increasing the movement speed, the derivation unit 32 derives a movement speed that has been adjusted to be higher for the movement speed of the patient table 3. Alternatively, in a case in which the operator voice corresponds to a voice that indicates decreasing the movement speed, the derivation unit 32 derives a movement speed that has been adjusted to be lower for the movement speed of the patient table 3.

As described above, in a case in which any information of the patient image, the biological information of the patient H, the patient voice, the operator image, or the operator voice acquired by the acquisition unit 30 during the movement of the patient table 3 satisfies a condition for adjusting the movement speed, the derivation unit 32 derives the adjusted movement speed according to the information. The adjustment of the movement speed is performed, for example, by increasing or decreasing the movement speed by a predetermined ratio such as 5% or a predetermined speed such as 1 [mm/s].

The movement control unit 34 controls the movement of the patient table 3 between the initial position P1 and the imaging position P2 in accordance with the movement speed derived by the derivation unit 32. Specifically, the movement control unit 34 outputs an instruction to start the movement to the driving unit 3C in accordance with the initial movement speed derived by the derivation unit 32. In response to the instruction, the driving unit 3C moves the patient table 3 from the initial position P1 toward the imaging position P2 at the initial movement speed.

In addition, the movement control unit 34 outputs an instruction to change the movement speed of the patient table 3 to the adjusted movement speed derived by the derivation unit 32 to the driving unit 3C during the movement of the patient table 3. In response to the instruction, the driving unit 3C changes the movement speed of the patient table 3.

Additionally, in a case in which the patient table 3 has reached the imaging position P2, the movement control unit 34 outputs an instruction to stop the patient table 3 to the driving unit 3C. In response to this instruction, the driving unit 3C stops the patient table 3. The movement control unit 34 may determine whether or not the patient table 3 has reached the imaging position P2 based on the optical image captured by the camera 7. Alternatively, the movement control unit 34 may determine whether or not the patient table 3 has reached the imaging position P2 based on an output of a position sensor that is provided on the patient table 3 to detect the position of the patient table 3. Further, the driving unit 3C may perform this determination. In this case, the movement control unit 34 need not output the instruction to stop the patient table 3 to the driving unit 3C.

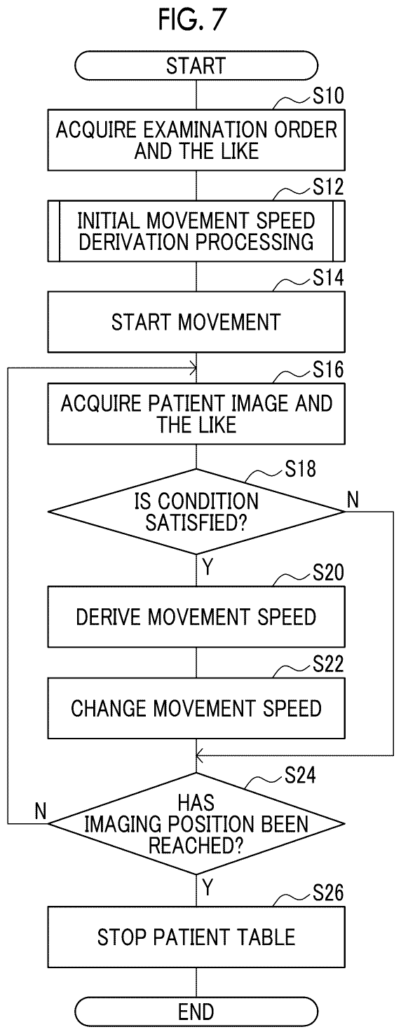

Next, an action of the information processing apparatus 10 will be described with reference to FIG. 7. The CPU 11 executes the information processing program 20 to execute the patient table movement processing shown in FIG. 7. The patient table movement processing is executed, for example, in a case in which an instruction to start execution is input by the operator.

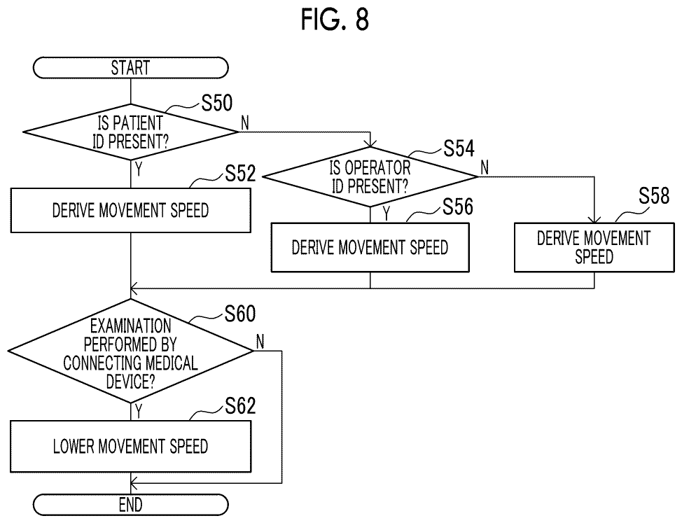

In step S10 of FIG. 7, the acquisition unit 30 acquires the examination order 22, the operator data 24, and the patient data 26 from the storage unit 13. In step S12, the initial movement speed derivation processing shown in FIG. 8 is executed.

In step S50 of FIG. 8, the derivation unit 32 determines whether or not the patient ID included in the examination order 22 is present in the patient data 26. In a case in which this determination results in an affirmative determination, the processing transitions to step S52. In step S52, the derivation unit 32 sets the movement speed associated with the patient ID included in the patient data 26 as the initial movement speed. In a case in which the processing in step S52 ends, the processing transitions to step S60.

In a case in which the determination in step S50 results in a negative determination, the processing transitions to step S54. In step S54, the derivation unit 32 determines whether or not the operator ID included in the examination order 22 is present in the operator data 24. In a case in which this determination results in an affirmative determination, the processing transitions to step S56. In step S56, the derivation unit 32 sets the movement speed associated with the operator ID included in the operator data 24 as the initial movement speed. In a case in which the processing in step S56 ends, the processing transitions to step S60.

In a case in which the determination in step S54 results in a negative determination, the processing transitions to step S58. In step S58, the derivation unit 32 sets a predetermined reference speed as the initial movement speed. In a case in which the processing of step S58 ends, the processing transitions to step S60.

In step S60, the derivation unit 32 determines whether or not the examination type included in the examination order 22 indicates an examination performed by connecting the medical device to the patient H. In a case in which this determination results in an affirmative determination, the processing transitions to step S62. In step S62, the derivation unit 32 lowers the initial movement speed derived in step S52, step S56, or step S58. In a case in which the processing of step S62 ends, the initial movement speed derivation processing ends. On the other hand, in a case in which the determination in step S60 results in a negative determination, the initial movement speed derivation processing ends without executing the processing of step S62.

In a case in which the initial movement speed derivation processing ends, step S12 of FIG. 7 ends, and the processing transitions to step S14. In step S14, the movement control unit 34 outputs an instruction to start the movement to the driving unit 3C in accordance with the initial movement speed derived in step S12. As a result, the movement of the patient table 3 from the initial position P1 toward the imaging position P2 is started.

In step S16, the acquisition unit 30 acquires the patient image, the biological information of the patient H, the patient voice, the operator image, and the operator voice. In step S18, as mentioned above, the derivation unit 32 determines whether or not any information of the patient image, the biological information of the patient H, the patient voice, the operator image, or the operator voice acquired in step S16 satisfies the condition for adjusting the movement speed. In a case in which this determination results in an affirmative determination, the processing transitions to step S20.

In step S20, as mentioned above, the derivation unit 32 derives the adjusted movement speed based on the information satisfying the condition in step S18 among the patient image, the biological information of the patient H, the patient voice, the operator image, and the operator voice. In step S22, the movement control unit 34 outputs an instruction to change the movement speed of the patient table 3 to the adjusted movement speed derived in step S20 to the driving unit 3C. In a case in which processing of step S22 ends, the processing transitions to step S24.

On the other hand, in a case in which the determination in step S18 results in a negative determination, the processing transitions to step S24 without executing the processing of step S20 and step S22. In step S24, the movement control unit 34 determines whether or not the patient table 3 has reached the imaging position P2. In a case in which this determination results in a negative determination, the processing returns to step S16, and in a case in which this determination results in an affirmative determination, the processing transitions to step S26. In step S26, the movement control unit 34 outputs an instruction to stop the patient table 3 to the driving unit 3C. In a case in which the processing of step S26 ends, the patient table movement processing ends.

In a case in which an instruction to start imaging is input by the operator after the above-described patient table movement processing had ended, the patient table portion 3A is slid with respect to the base portion 3B, and the patient H lying on the patient table portion 3A is transported into the opening portion 5 of the gantry 2, and the imaging of the CT image is started.

As described above, according to the present embodiment, the movement speed of the patient table on which the subject is placed in the medical image capturing apparatus can be controlled to an appropriate speed.

In the above-described embodiment, a case in which the disclosed technology is applied to the control of the movement speed of the patient table 3 from the initial position P1 to the imaging position P2 before the image capturing by the CT apparatus 1 has been described, but the disclosed technology is not limited to this aspect. A form may be employed in which the disclosed technology is applied to control of the movement speed of the patient table 3 from the imaging position P2 to the initial position P1 after the image capturing by the CT apparatus 1.

In addition, in the above-described embodiment, a case in which the CPU 11 specifies the operator of the CT apparatus 1 based on the inclusion of the operator ID in the examination order 22 has been described, but the disclosed technology is not limited to this aspect. For example, a form may be employed in which the CPU 11 specifies the operator based on a signal transmitted from an integrated circuit (IC) tag worn by the operator. Alternatively, for example, a form may be employed in which the CPU 11 specifies the operator based on voiceprint information of the voice uttered by the operator. Further, for example, a form may be employed in which the CPU 11 specifies the operator by performing image analysis processing on the image captured by the camera 7.

Additionally, in the above-described embodiment, a case in which the CPU 11 specifies the patient H to be examined using the CT apparatus 1 based on the inclusion of the patient ID in the examination order 22 has been described, but the disclosed technology is not limited to this aspect. For example, a form may be employed in which the CPU 11 specifies the patient H based on a signal transmitted from an IC tag worn by the patient H. Alternatively, for example, a form may be employed in which the CPU 11 specifies the patient H based on voiceprint information of the voice uttered by the patient H. Further, for example, a form may be employed in which the CPU 11 specifies the patient H by performing image analysis processing on the image captured by the camera 7.

In addition, in the above-described embodiment, the derivation unit 32 may derive the initial movement speed based on a disease from which the patient H is suffering, the treatment status of the disease, the medication information of the patient H, and the like. For example, in a case in which the patient H is suffering from a specific disease, the derivation unit 32 may derive the movement speed of the patient table 3 to be lower than in a case in which the patient H is not suffering from the specific disease.

Additionally, in the above-described embodiment, a case in which the CPU 11 determines whether or not the examination type is an examination performed by connecting the medical device to the patient H based on the examination type included in the examination order 22 has been described, but the disclosed technology is not limited to this aspect. For example, the CPU 11 may determine whether or not the medical device is connected to the patient H by performing image analysis processing on the patient image.

Further, in the above-described embodiment, in a case in which an abnormality such as an earthquake, a falling object, or smoke is detected in the imaging room, the CPU 11 may perform control to stop the movement of the patient table 3 and may issue a notification that the abnormality has been detected.

Furthermore, at least one of functional units provided in the information processing apparatus 10 in the above-described embodiment may be provided in other devices such as a control device provided in the gantry 2 and a control device provided in the driving unit 3C.

Moreover, in the above-described embodiment, for example, as a hardware structure of a processing unit that executes various kinds of processing such as each functional unit of the information processing apparatus 10, the following various processors can be used. The above-described various processors include, in addition to the CPU which is a general-purpose processor that executes software (programs) to function as various processing units as mentioned above, a programmable logic device (PLD) which is a processor having a changeable circuit configuration after manufacture such as an FPGA, a dedicated electrical circuit which is a processor having a dedicated circuit configuration designed to execute specific processing such as an application specific integrated circuit (ASIC), and the like.

One processing unit may be configured with one of these various processors or may be configured with a combination of two or more processors of the same type or different types (for example, a combination of a plurality of FPGAs or a combination of a CPU and an FPGA). Alternatively, a plurality of processing units may be configured with one processor.

As an example of configuring a plurality of processing units with one processor, first, there is an aspect in which one or more CPUs and software are combined to constitute one processor and the processor functions as the plurality of processing units, as represented by a computer such as a client and a server. Secondly, there is an aspect in which a processor that implements the functions of the entire system, which includes a plurality of processing units, with one integrated circuit (IC) chip is used, as represented by a system on chip (SoC) or the like. In this manner, the various processing units are configured using one or more of the above-described various processors as the hardware structure.

Furthermore, as the hardware structure of these various processors, more specifically, an electrical circuit (circuitry) in which circuit elements such as semiconductor elements are combined can be used.

In addition, in the above-described embodiment, an aspect in which the information processing program 20 is stored (installed) in advance in the storage unit 13 has been described, but the disclosed technology is not limited to this aspect. The information processing program 20 may be provided in a form recorded on a recording medium such as a compact disc read only memory (CD-ROM), a digital versatile disc read only memory (DVD-ROM), and a universal serial bus (USB) memory. Alternatively, the information processing program 20 may be provided in a form that can be downloaded from an external device via a network.

What is claimed is:

1. An information processing apparatus that derives a movement speed of a patient table of a medical image capturing system which captures a medical image of a subject placed on the patient table, the information processing apparatus comprising:
at least one processor,
wherein the processor is configured to:
acquire at least one of examination information regarding an examination, subject information regarding the subject, or operator information regarding an operator; and
derive a movement speed of the patient table between an initial position and an imaging position based on the acquired information.

2. The information processing apparatus according to claim 1,
wherein the processor is configured to:
control movement of the patient table between the initial position and the imaging position in accordance with the derived movement speed.

3. The information processing apparatus according to claim 1,
wherein the examination information includes a type of examination.

4. The information processing apparatus according to claim 3,
wherein the processor is configured to, in a case in which the type of examination indicates an examination performed by connecting a medical device to the subject, derive the movement speed of the patient table to be lower than in a case in which the type of examination indicates an examination performed without connecting the medical device to the subject.

5. The information processing apparatus according to claim 1,
wherein the subject information includes at least one of information associated with identification information of the subject, biological information of the subject, a voice uttered by the subject placed on the patient table, or an optical image obtained by imaging the subject placed on the patient table.

6. The information processing apparatus according to claim 5,
wherein the processor is configured to:
derive a movement speed of the patient table before start of movement of the patient table and then derive a movement speed that has been adjusted for the derived movement speed of the patient table based on at least one of the biological information, the voice, or the optical image acquired during the movement of the patient table.

7. The information processing apparatus according to claim 1,
wherein the operator information includes at least one of information associated with identification information of the operator, a voice uttered by the operator, or an optical image obtained by imaging the operator.

8. The information processing apparatus according to claim 7,
wherein the processor is configured to:
derive a movement speed of the patient table before start of movement of the patient table and then derive a movement speed that has been adjusted for the derived movement speed of the patient table based on at least one of the voice or the optical image acquired during the movement of the patient table.

9. An information processing method comprising:
causing a processor of an information processing apparatus that includes at least one processor and that derives a movement speed of a patient table of a medical image capturing system which captures a medical image of a subject placed on the patient table, to execute:
acquiring at least one of examination information regarding an examination, subject information regarding the subject, or operator information regarding an operator; and
deriving a movement speed of the patient table between an initial position and an imaging position based on the acquired information.

10. A non-transitory computer-readable storage medium storing an information processing program for causing a processor of an information processing apparatus that includes at least one processor and that derives a movement speed of a patient table of a medical image capturing system which captures a medical image of a subject placed on the patient table, to execute:
acquiring at least one of examination information regarding an examination, subject information regarding the subject, or operator information regarding an operator; and
deriving a movement speed of the patient table between an initial position and an imaging position based on the acquired information.
